(19) United States
(12) Patent Application Publication
(10) Pub. No.: US 2009/0281129 A1
(43) Pub. Date: Nov. 12, 2009

(54) CDKI PATHWAY INHIBITORS AND USES THEREOF

(75) Inventors: Bey-Dih Chang, MADISON, WI (US); Igor B. Roninson, Loudonville, NY (US); Donald Porter, Middle Grove, NY (US)

Correspondence Address: KEOWN & ZUCCHERO, LLP, 500 WEST CUMMINGS PARK, SUITE 1200, WOBURN, MA, 01801, US

(73) Assignee: Senex Biotechnology, Inc.

(21) Appl. No.: 12/187,536

(22) Filed: Aug. 7, 2008

Related U.S. Application Data
(63) Continuation-in-part of application No. 11/803,693, filed on May 15, 2007.
(60) Provisional application No. 60/747,213, filed on May 15, 2006.

(51) Int. Cl. A61K 31/517; A61P 25/28

(52) U.S. Cl. 514/266.4

(57) ABSTRACT

The invention relates to the inhibition of the Cyclin-Dependent Kinase Inhibitor (CDKI) pathway. More particularly, the invention relates to methods for inhibiting the CDKI pathway for studies of and intervention in senescence-related and other CDKI-related diseases.

This application is a continuation-in-part of Ser. No. 11/803,693, filed May 15, 2007 and claims priority from U.S. provisional application 60/747,213, filed May 15, 2006.

### BACKGROUND OF THE INVENTION

1. Field of the Invention

The invention relates to the inhibition of the Cyclin-Dependent Kinase Inhibitor (CDKI) pathway. More particularly, the invention relates to methods for inhibiting the CDKI pathway for studies of and intervention in senescence-related diseases, including degenerative diseases of the central nervous system, including Alzheimer's Disease and other dementias.

2. Summary of the Related Art

Cell senescence, originally defined as a series of cellular changes associated with aging, is now viewed more broadly as a signal transduction program leading to irreversible cell cycle arrest, accompanied by a distinct set of changes in the cellular phenotype (See e.g. Campisi, Cell 120: 513-522 (2005); Shay and Roninson, Oncogene 23: 2919-2933 (2004)). Senescence can be triggered by many different mechanisms including the shortening of telomeres (replicative senescence) or by other endogenous and exogenous acute and chronic stress signals, including major environmental factors, such as UV and cigarette smoke. The latter forms of telomere-independent senescence are variably referred to as accelerated senescence, STASIS (Stress or Aberrant Signaling Induced Senescence), or SIPS (Stress-Induced Premature Senescence). Regardless of the mode of induction, senescent cells develop the same general phenotype, characterized not only by permanent growth arrest but also by enlarged and flattened morphology, increased granularity, high lysosomal mass, and expression of senescence-associated endogenous β-galactosidase activity (SA-β-gal).

Dimri et al., Proc. Natl. Acad. Sci. USA 92: 9363-9367 (1995) teaches that in the human body, the phenotype of cell senescence has been detected in correlation with aging. Castro et al., Prostate 55: 30-38 (2003); Michaloglou et al., Nature 436: 720-724 (2005); and Collado et al., Nature 436: 642 (2005) teach that the phenotype of cell senescence has also been detected in pathological situations, including various pre-malignant conditions. te Poele et al., Cancer Res. 62: 1876-1883 (2002); and Roberson et al., Cancer Res. 65: 2795-2803 (2005) teach its detection in many tumors treated with chemotherapy.

In most systems of senescence that have been characterized at the molecular level, cell cycle arrest is triggered by the activation of p53, which in its turn induces a broad-specificity cyclin-dependent kinase inhibitor (CDKI) p21^{Wafl/CiP1/Sdi1}. p21 induction causes cell cycle arrest at the onset of senescence, but p53 and p21 levels decrease at a later stage. Shay and Roninson, Oncogene 23: 2919-2933 (2004) teach that this decrease is accompanied, however, by a stable increase in another CDKI protein, p16^{Ink4A}, which is believed to be primarily responsible for the maintenance of cell cycle arrest in senescent normal cells.

CDKI proteins act as negative regulators of the cell cycle and are therefore generally known as tumor suppressors. The induction of CDKI proteins, in particular p21, also occurs in tumor cells in the context of cancer therapy, in response to cellular damage by different classes of cancer chemotherapeutic drugs and ionizing radiation. Cell cycle arrest by CDKIs mediates the cytostatic and senescence-inducing activity of anticancer agents, one of the major components of their therapeutic effect (Roninson, Cancer Res., 11, 2705-2715). Agents that would enhance the ability of CDKI proteins to induce cell cycle arrest will therefore be useful for the chemoprevention of cancer and for increasing the therapeutic efficacy of conventional anticancer agents.

Although senescent cells do not divide, they remain fully viable, metabolically and synthetically active. It has now been recognized that senescent cells secrete a variety of factors that have a major effect on their environment. Campisi, supra teaches that secretory activities of senescent cells have been linked to carcinogenesis, skin aging, and a variety of age-related diseases. A series of studies have implicated p21 and other CDKI proteins in disease-promoting activities of senescent cells. This insight came principally from the analysis by Chang et al., Proc. Natl. Acad. Sci. USA 97: 4291-4296 (2000) of the transcriptional effects of p21, expressed in a fibroblastoid cell line from an inducible promoter. This analysis showed that p21 produces significant changes in the expression of multiple genes. Many genes are strongly and rapidly inhibited by p21, and most of these are involved in cell proliferation. Zhu et al., Cell Cycle 1: 50-58 (2002) teaches that inhibition of cell cycle progression genes by p21 is mediated by negative cis-regulatory elements in the promoters of these genes, such as CDE/CHR. The same genes are downregulated in tumor cells that undergo senescence after chemotherapeutic treatment, but Chang et al., Proc. Natl. Acad. Sci. USA 99: 389-394 (2002) teaches that p21 knockout prevents the inhibition of these genes in drug-treated cells. Hence, p21 is responsible for the inhibition of multiple cell cycle progression genes in response to DNA damage.

Chang et al., 2000, supra teaches that another general effect of p21 induction is upregulation of genes, many of which encode transmembrane proteins, secreted proteins and extracellular matrix (ECM) components. This effect of p21 is relatively slow, occurring subsequently to growth arrest and concurrently with the development of the morphological features of senescence. These genes are induced by DNA damage but p21 knockout decreases their induction (Chang et al., 2002, supra). This decrease is only partial, which can be explained by recent findings by that the majority of p21-inducible genes are also induced in response to other CDKI, p16 and p27 (see WO 03/073062). Gregory et al., Cell Cycle 1: 343-350 (2002); and Poole et al., Cell Cycle 3: 931-940 (2004) teach that gene upregulation by CDKI has been reproduced using promoter constructs of many different CDKI-inducible genes, indicating that it occurs at the level of transcription. (Perkins et al., Science 275: 523-527 (1997); Gregory et al., supra; and Poole et al., supra teach that induction of transcription by p21 is mediated in part by transcription factor NFκB and transcription cofactors of p300/CBP family, but other intermediates in the signal transduction pathway that leads to the activation of transcription in response to CDKI—the CDKI pathway—remain presently unknown (FIG. 1).

Medical significance of the induction of transcription by CDKI has been indicated by the known functions of CDKI-inducible genes (Chang et al., 2000, supra). Many CDKI-upregulated genes are associated with cell senescence and organism aging, including a group of genes implicated in age-related diseases and lifespan restriction. One of these genes is p66^{She}, a mediator of oxidative stress, the knockout of which expands the lifespan of mice by about 30% (Migliaccio et al., supra). Many CDKI-induced genes play a role in age-related diseases, most notably Alzheimer's disease and amyloidosis. Thus, CDKI induce many human amyloid proteins, including Alzheimer's amyloid β precursor protein (βAPP) and serum amyloid A, implicated in amyloidosis, atherosclerosis and arthritis. CDKI also upregulate tissue transglutaminase that cross-links amyloid peptides leading to plaque formation in both Alzheimer's disease and amyloidosis. Some of CDKI-inducible genes are connective tissue growth factor and galectin-3 involved in atherosclerosis, as well as cathepsin B, fibronectin and plasminogen activator inhibitor 1, associated with arthritis. Murphy et al., J. Biol. Chem. 274: 5830-5834 (1999) teaches that several CDKI-inducible proteins are also implicated in an in vitro model of nephropathy. Remarkably, p21-null mice were found to be resistant to experimental induction of atherosclerosis (Merched and Chan, Circulation 110: 3830-3841 (2004)) and chronic renal disease (Al Douahji et al., Kidney Int. 56: 1691-1699 (1999); Megyesi et al., Proc. Natl. Acad. Sci. USA 96: 10830-10835 (1999).

In addition to their effect on cellular genes, CDKI stimulate the promoters of many human viruses, such as HIV-1, cytomegalovirus, adenovirus and SV40. Since many viruses induce p21 expression in infected cells, this effect suggests that promoter stimulation by CDKI may promote viral infections (Poole et al., supra).

Strong associations for CDKI-inducible genes have also been found in cancer. In particular, p21 expression activates the genes for many growth factors, inhibitors of apoptosis, angiogenic factors, and invasion-promoting proteases. In accordance with these changes in gene expression, Chang et al., 2000, supra teaches that p21-arrested cells show paracrine mitogenic and anti-apoptotic activities in coculture assays. Krtolica et al., Proc. Natl. Acad. Sci. USA 98: 12072-12077 (2001) teaches that paracrine tumor-promoting activities were demonstrated both in vitro and in vivo in CDKI-expressing normal senescent fibroblasts, which express p21 and p16. Importantly, senescent fibroblasts possess the characteristic pro-carcinogenic activity that has long been identified with tumor-associated stromal fibroblasts. Furthermore, all the experimental treatments shown to endow fibroblasts with tumor-promoting paracrine activities also induce CDKI, suggesting that the CDKI pathway could be the key mediator of pro-carcinogenic activity of stromal fibroblasts (Roninson, Cancer Lett. 179: 1-14 (2002)).

CDKI expression mediates cell cycle arrest not only in the program of senescence but also in numerous other situations, such as transient checkpoint arrest in response to different forms of damage, contact inhibition, and terminal differentiation. Hence, the CDKI pathway, which leads to the activation of multiple disease-promoting genes, is activated not only in cell senescence but also in many other physiological situations. As a result, CDKI-responsive gene products are expected to accumulate over the lifetime, contributing to the development of Alzheimer's disease, amyloidosis, atherosclerosis, arthritis, renal disease and cancer.

There is, therefore, a need for methods for inhibiting the CDKI pathway which may have a variety of clinical applications in chemoprevention and therapy of different age-related diseases. Useful CDKI pathway inhibitors should not interfere with the function of CDKI proteins as inhibitors of the cell cycle but rather inhibit the key signal transduction events that lead to the induction of transcription of CDKI-responsive genes. The ideal CDKI pathway inhibitors should both inhibit the CDKI pathway and enhance the tumor-suppressive cell cycle-inhibitory activity of the CDKI proteins.

### BRIEF SUMMARY OF THE INVENTION

The invention provides pharmaceutical formulations and methods for treating degenerative diseases of the central nervous system, including Alzheimer's Disease and other dementias.

The invention provides methods for inhibiting the induction of transcription by the Cyclin-Dependent Kinase Inhibitor (CDKI) pathway. A high throughput screening system, described in greater detail in application number PCT/US06/01046, has been used to screen over 100,000 drug-like small molecules from commercially available diversified compound collections. Through this screening, the present inventors have identified a set of active compounds. These include a series of structurally related compounds, which inhibit the induction of all the tested genes by CDKI and also reverse CDKI-induced transcription. These molecules, identified herein as SNX2-class compounds, show little or no cytotoxicity in normal cells. These molecules do not interfere with the cell cycle-inhibitory function of CDKIs and even enhance the induction of G1 cell cycle arrest by CDKI proteins. SNX2-class compounds block the development of the senescent morphology in fibroblasts arrested by DNA damage. They also inhibit the secretion of anti-apoptotic factors by CDKI-arrested cells. The invention has demonstrated the feasibility of blocking the disease-promoting CDKI pathway without interfering with the essential tumor-suppressing function of CDKI. The molecules discovered according to the invention provide a lead family of compounds with this promising biological activity.

The invention provides methods for enhancing induction of G1 cell cycle arrest by CDKI proteins comprising contacting a cell with a compound that enhances the induction of G1 cell cycle arrest by CDKI proteins. In some preferred embodiments, the cell cycle-inhibitory activity of CDKI proteins is mediated by the inhibition of CDK2. The enhancement of the induction of G1 cell cycle arrest by CDKI proteins can be used for the chemoprevention and treatment of cancer and other diseases associated with abnormal cell proliferation and for increasing the ability of CDKI-inducing cancer therapeutic agents to arrest the growth of cancer cells. In certain embodiments the method according to the invention comprises contacting a cell with a small molecule compound having the structure (I). In certain embodiments, the small molecule has a structure selected from the group of compounds shown in FIG. 2. In some preferred embodiments, the cell cycle-inhibitory activity of CDKI proteins is mediated by the inhibition of CDK2.

The invention also provides methods for stimulating the cell cycle-inhibitory activity of CDKI proteins using compounds that inhibit the induction of transcription by the CDKI pathway. Particularly preferred are methods that utilize compounds having Structure I, including without limitation the compounds shown in FIG. 2.

The invention further provides methods for identifying a compound that enhances induction of G1 cell cycle arrest by CDKI proteins, the method comprising (i) expressing a CDKI protein in a cell at a level that induces sub-maximal G1 arrest, (ii) contacting the cell with a test compound, (iii) measuring the extent of G1 arrest in the presence and in the absence of a test compound, wherein the test compound is identified as a compound that enhances induction of G1 cell cycle arrest by CDKI proteins if the test compound increases the extent of G1 arrest. For purposes of the invention, “sub-maximal G1 arrest” means arrest in G1 phase of an adequate number of cells to allow the observation in the increase in the numbers of cells in G1 phase in the presence of a CDKI protein versus the number of cells in G1 phase in the absence of the CDKI protein.

The invention further provides methods for identifying a compound that is useful as a therapeutic for a CDKI-mediated disease (including but not limited to Alzheimer's disease, atherosclerosis, amyloidosis, arthritis, chronic renal disease, viral diseases and cancer), the method comprising contacting a cell with a test compound, measuring the ability of the test compound to inhibit the Cyclin-Dependent Kinase Inhibitor (CDKI) pathway, contacting a cell with a second compound having the structure of a compound useful in the first aspect of the invention, measuring the ability of the second compound to inhibit the Cyclin-Dependent Kinase Inhibitor (CDKI) pathway; and comparing the ability of the test compound and the second compound to inhibit the Cyclin-Dependent Kinase Inhibitor (CDKI) pathway; wherein the test compound is identified as a compound that is useful as a therapeutic for a CDKI-mediated disease if the test compound has an ability equal to or better than the second compound to inhibit the Cyclin-Dependent Kinase Inhibitor (CDKI) pathway. This aspect of the invention further provides compounds identified according to this method.

In addition, the invention provides a method for therapeutically treating a mammal having a CDKI-mediated disease comprising administering to the mammal a therapeutically effective amount of a compound that is useful in the methods according to the first and second aspect of the invention.

### BRIEF DESCRIPTION OF THE DRAWINGS

FIG. 1 shows the structures of 56 compounds effective in the inhibition of the signal transduction pathway that leads to the activation of transcription in response to CDKI.

FIG. 2 shows the structure of active compounds of SNX2 family that inhibit the signal transduction pathway that leads to the activation of transcription in response to CDKI.

FIG. 3 shows the structure of inactive compounds of SNX2 family.

FIG. 4 shows the effects of different doses of some SNX2-class compounds on CMV promoter activity, represented as GFP expression in a reporter cell line from the CMV promoter normalized by cellular DNA content (a measure of cell number) as measured by Hoechst 33342 staining, in the presence or in the absence of IPTG (the p21 inducer).

FIG. 5 shows that SNX38 not only prevents but also reverses p21-induced transcription.

FIG. 6 shows the data obtained with SNX2 and SNX14 in p21-arrested cells, with the results expressed as the ratio of RNA levels for each gene in the presence and in the absence of IPTG.

FIG. 7 shows the data obtained with SNX2 and SNX14 in p16 arrested cells, with the results expressed as the ratio of RNA levels for each gene in the presence and in the absence of IPTG.

FIG. 8 shows that SNX2 does not inhibit binding of NFκB proteins p50 or p65 to double-stranded DNA oligonucleotide comprising NFκB binding site. Each set shows oligonucleotide binding to p50 in control cells (left bars) and in cells treated with known NF-κB inducer TNFα (second bars), as well as oligonucleotide binding to p65 in control (third bars) or TNFα-treated cells (right bars). The left set of bars represents cells treated with carrier control, the middle set represents cells treated with SNX2, and the right set represents cells treated with a known inhibitor of NFκB binding (TPCK).

FIG. 9 shows FACS analysis of DNA content in DAPI-stained HT1080 p21-9 cells, which were either untreated or treated for 18 hrs with 20 μM SNX2 or SNX14, in the absence or in the presence of 50 μM IPTG.

FIG. 10 shows changes in the G1, S and G2/M fractions of HT1080 p27-2 cells (as determined by FACS analysis of DNA content), upon 24-hour treatment with the indicated concentrations of IPTG, in the absence of SNX14, or in the presence of 20 μM or 40 μM of SNX14.

FIG. 11 shows that doxorubicin induces expression of the senescence marker SA-β-gal (blue staining), but SNX2 and SNX14 block this phenotype.

FIG. 12 shows results of an assay for paracrine antiapoptotic activity of p21-expressing HT1080 p21-9 cells, as measured by the survival of C8 cells in low-serum media, in which HT 1080 p21-9 cells were either untreated or treated with p21-inducing IPTG, alone or in the presence of SNX2-class compounds (SNX2, SNX14 or SNX38).

### DETAILED DESCRIPTION OF THE PREFERRED EMBODIMENTS

The invention relates to the inhibition of the Cyclin-Dependent Kinase Inhibitor (CDKI) pathway. More particularly, the invention relates to methods for inhibiting the CDKI pathway for studies of and intervention in senescence-related diseases. The patents and publications cited herein reflect the level of knowledge in this field and are hereby incorporated by reference in their entirety. Any conflict between the teachings of the cited references and this specification shall be resolved in favor of the latter.

The invention provides pharmaceutical formulations and methods for treating degenerative diseases of the central nervous system, including Alzheimer's Disease and other dementias.

The invention provides methods for inhibiting the CDKI pathway which may have a variety of clinical applications in chemoprevention and therapy of different age-related diseases. The CDKI pathway inhibition methods according to the invention utilize molecules, identified herein as SNX2-class compounds, that show little or no cytotoxicity in normal cells. These molecules do not interfere with the cell cycle-inhibitory function of CDKIs and even enhance the induction of G1 cell cycle arrest by CDKI proteins. SNX2-class compounds block the development of the senescent morphology in fibroblasts arrested by DNA damage. They also inhibit the secretion of anti-apoptotic factors by CDKI-arrested cells. The invention has demonstrated the feasibility of blocking the disease-promoting CDKI pathway without interfering with the essential tumor-suppressing function of CDKI. The molecules discovered according to the invention provide a lead family of compounds with this promising biological activity.

In a first aspect, the invention provides methods for enhancing induction of G1 cell cycle arrest by CDKI proteins comprising contacting a cell with a compound that enhances the induction of G1 cell cycle arrest by CDKI proteins. In some preferred embodiments, the cell cycle-inhibitory activity of CDKI proteins is mediated by the inhibition of CDK2. The enhancement of the induction of G1 cell cycle arrest by CDKI proteins can be used for the chemoprevention and treatment of cancer and other diseases associated with abnormal cell proliferation and for increasing the ability of CDKI-inducing cancer therapeutic agents to arrest the growth of cancer cells.

In preferred embodiments, the method according to the invention comprises contacting a cell with a small molecule inhibitor having the structure (I):

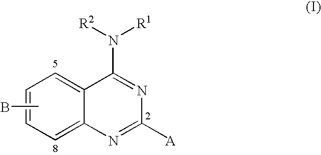

wherein
R¹ is selected from lower alkyl, cycloalkyl, alkenyl, alkynyl, hydroxyalkyl, alkoxyalkyl, hydroxyalkoxyalkyl, dialkylaminoalkyl, aralkyl, aryl, heteroaryl, phenethyl, and alkoxyphenyl;
R² is selected from R¹ and hydrogen;
A is selected from hydrogen or R¹; and
B is halogen.
In certain preferred embodiments, R¹ is selected from C1-C3 alkyl, C2-C3 alkenyl, C2-C3 alkynyl, C7-C8 aralkyl, C2-C3-O-alkyl substituted aryl, and a 3-6 membered heteroalkyl group having 1-2 heteroatoms selected from O and N, wherein R¹ is C2-C3 alkyl when R² is not hydrogen.
In certain embodiments, R² is preferably hydrogen. In certain preferred embodiments, A is hydrogen.

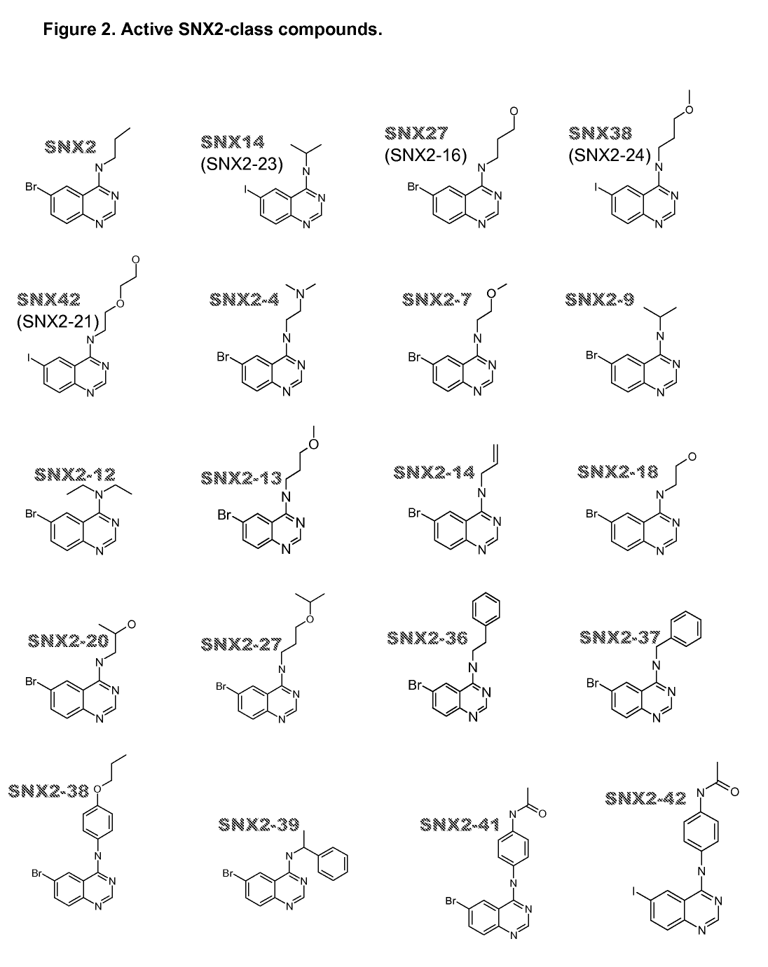

In certain preferred embodiments, the small molecule has a structure selected from the group of structures shown in FIG. 2.

In a second aspect, the invention provides methods for stimulating the cell cycle-inhibitory activity of CDKI proteins using compounds that inhibit the induction of transcription by the CDKI pathway. For purposes of the invention, “inhibiting the induction of transcription by the CDKI pathway” means either preventing or reducing induction of transcription by the CDKI pathway in the presence of a compound according to the invention relative to in the absence of the compound, or reducing such induction that has already occurred, using the compound, relative to the absence of the compound. As a practical measure of the method according to this aspect of the invention, the method should not inhibit the essential tumor-suppressive role of CDKI proteins, nor should it directly inhibit the function of proteins encoded by genes that are transcriptionally activated by the CDKI pathway. However, inhibition of transcription of genes that are transcriptionally activated by the CDKI pathway is not regarded as direct inhibition of the function of proteins encoded by genes that are transcriptionally activated by the CDKI pathway. Particularly preferred are methods that utilize compounds having Structure I, including without limitation the compounds shown in FIG. 2.

In a third aspect the invention provides methods for identifying a compound that enhances induction of G1 cell cycle arrest by CDKI proteins, the method comprising (i) expressing a CDKI protein in a cell at a level that induces sub-maximal G1 arrest, (ii) contacting the cell with a test compound, (iii) measuring the extent of G1 arrest in the presence and in the absence of a test compound, wherein the test compound is identified as a compound that enhances induction of G1 cell cycle arrest by CDKI proteins if the test compound increases the extent of G1 arrest. For purposes of the invention, “sub-maximal G1 arrest” means arrest in G1 phase of an adequate number of cells to allow the observation in the increase in the numbers of cells in G1 phase in the presence of a CDKI protein versus the number of cells in G1 phase in the absence of the CDKI protein. The actual number of cells fitting this description will vary depending on the cell line, the CDKI protein, and the conditions for expressing the CDKI protein. However, for any cell line and CDKI expression system this number can be readily determined empirically, as described in the examples below.

In particular, Example 4 illustrates the use of a regulated promoter system to express a CDKI protein in a mammalian cell at an intermediate level, which induces G1 arrest to a sub-maximal extent. Alternatively, intermediate levels of CDKI expression can be achieved by transfecting cells with different amounts of a vector that expresses a CDKI protein, or by delivering different amounts of a CDKI protein into cells directly using a suitable delivery vehicle, such as a liposome. In another alternative approach, the ability of a compound to enhance CDKI-induced G1 arrest may be identified in a cell-free system, by measuring the effect of a purified CDKI protein on the kinase activity of a cyclin/CDK complex, in the presence or in the absence of a test compound, and identifying the test compound as enhancing induction of G1 cell cycle arrest by CDKI proteins if the kinase activity is inhibited by the CDKI protein to a greater extent in the presence of the compound than in the absence of the compound. In preferred embodiments, the cyclin/CDK complex comprises CDK2 and a CDK2-interacting cyclin, and the CDKI protein comprises p21 or p27.

In a fourth aspect, the invention provides methods for identifying a compound that is useful as a therapeutic for a CDKI-mediated disease (including but not limited to Alzheimer's disease, atherosclerosis, amyloidosis, arthritis, chronic renal disease, viral diseases and cancer), the method comprising contacting a cell with a test compound, measuring the ability of the test compound to inhibit the Cyclin-Dependent Kinase Inhibitor (CDKI) pathway, contacting a cell with a second compound having the structure of a compound useful in the first aspect of the invention, measuring the ability of the second compound to inhibit the Cyclin-Dependent Kinase Inhibitor (CDKI) pathway; and comparing the ability of the test compound and the second compound to inhibit the Cyclin-Dependent Kinase Inhibitor (CDKI) pathway; wherein the test compound is identified as a compound that is useful as a therapeutic for a CDKI-mediated disease if the test compound has an ability equal to or better than the second compound to inhibit the Cyclin-Dependent Kinase Inhibitor (CDKI) pathway. This aspect of the invention further provides compounds identified according to this method.

In a fifth aspect of the invention, the invention provides a method for therapeutically treating a mammal having a CDKI-mediated disease comprising administering to the mammal a therapeutically effective amount of a compound that is useful in the methods according to the first and second aspect of the invention.

The results herein demonstrate that SNX2-class compounds exhibit all the essential biological effects expected for CDKI pathway inhibitors, as they block the induction of disease-associated gene expression, paracrine antiapoptotic activities, and the senescent phenotype of CDKI-arrested cells. Thus, the invention provides SNX2-class compounds which therefore constitute prototypes of drugs that are likely to be useful for chemoprevention or therapy of Alzheimer's disease, amyloidosis, atherosclerosis, renal disease, viral diseases, or cancer.

### Pharmaceutical Formulations and Administration

In the methods according to the invention, the compounds described above may be incorporated into a pharmaceutical formulation. Such formulations comprise the compound, which may be in the form of a free acid, salt or prodrug, in a pharmaceutically acceptable diluent, carrier, or excipient. Such formulations are well known in the art and are described, e.g., in Remington's Pharmaceutical Sciences, 18th Edition, ed. A. Gennaro, Mack Publishing Co., Easton, Pa., 1990.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art.

As used herein, the term pharmaceutically acceptable salts refers to salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art. In certain applications, including without limitation, senile dementias such as Alzheimer's, an effective dose range for a 70 kg patient is from about 50 mg per patient per day up to about 10 grams per patient per day, or the maximum tolerated dose. In certain preferred embodiments the dose range is from about 200 mg per patient per day to about 10 g per patient per day. In certain preferred embodiments the dose range is from about 200 mg per patient per day to about 5 g per patient per day. The dose in each patient may be adjusted depending on the clinical response to the administration of a particular drug.

Administration of the pharmaceutical formulations in the methods according to the invention may be by any medically accepted route, including, without limitation, parenteral, oral, sublingual, transdermal, topical, intranasal, intratracheal, or intrarectal. In certain preferred embodiments, compositions of the invention are administered parenterally, e.g., intravenously in a hospital setting. In certain other preferred embodiments, administration may preferably be by the oral route.

The following examples are intended to further illustrate certain preferred embodiments of the invention and are not intended to limit the scope of the invention.

### Example 1

### Identification of CDKI Pathway Inhibitors

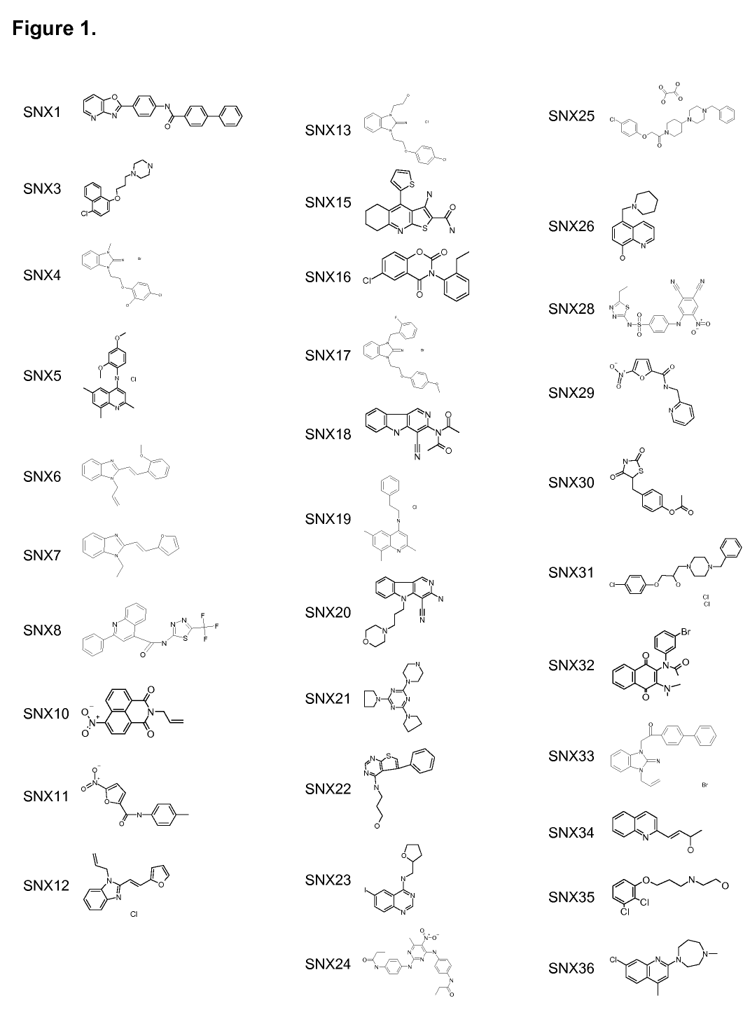

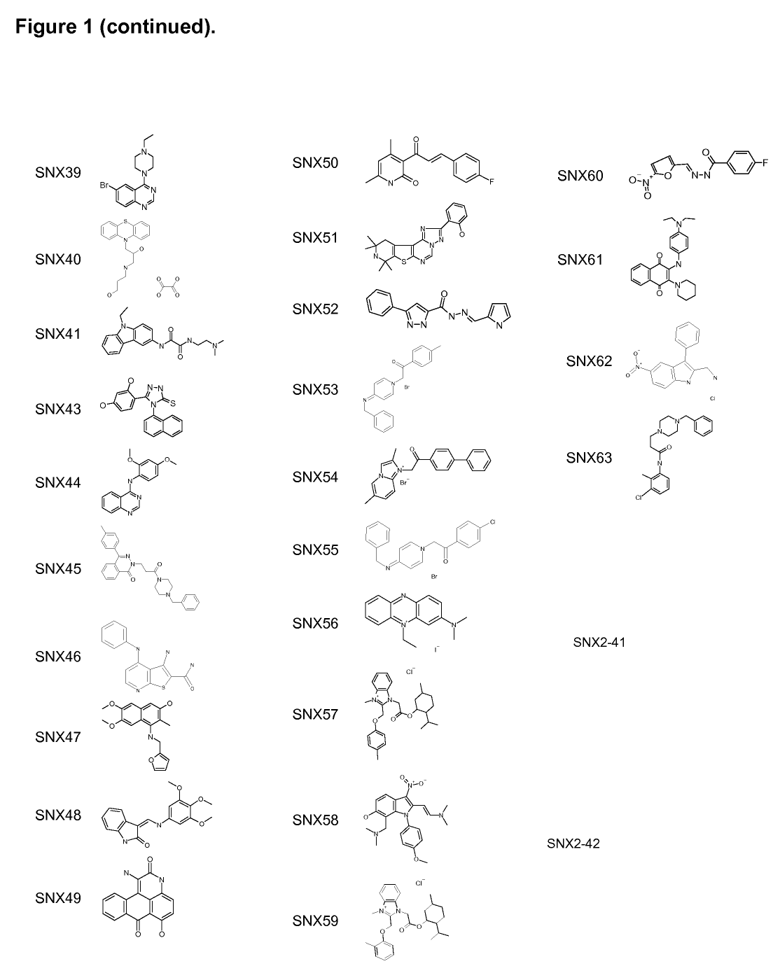

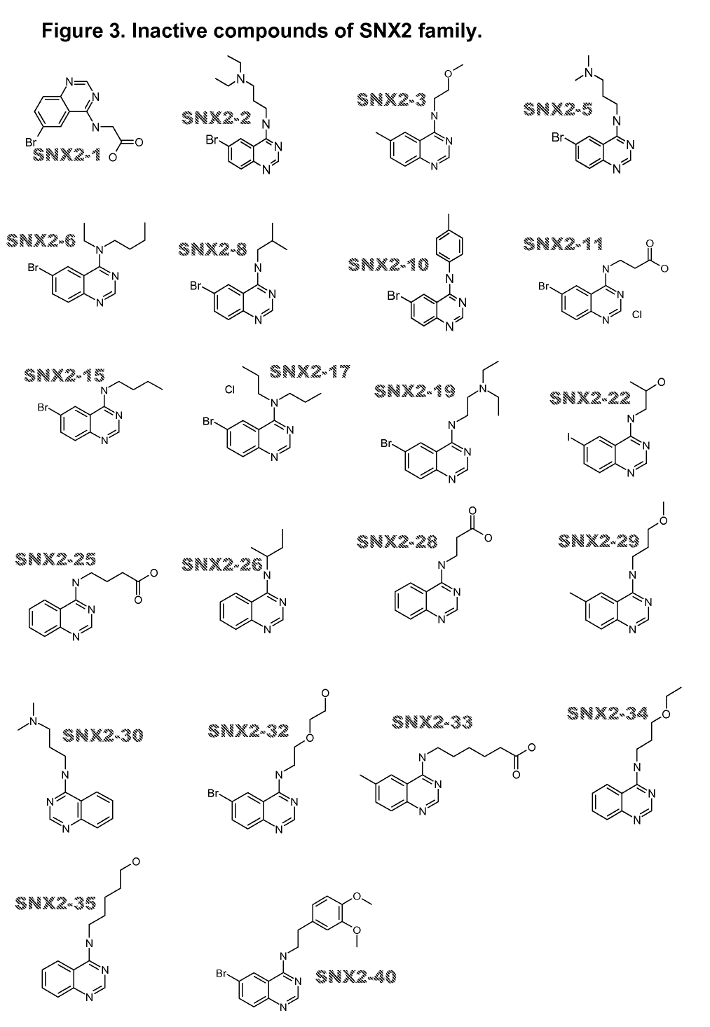

The present inventors have developed a high-throughput screening (HTS) procedure for compounds inhibiting the CDKI pathway. This procedure utilizes a highly sensitive reporter cell line that was generated by infecting HT1080 p21-9 cells, a derivative of HT 1080 fibrosarcoma cells that express p21 from a promoter induced by a physiologically neutral β-galactoside IPTG (isopropyl-β-thio-galactoside) with a lentiviral vector that expresses Green Fluorescent Protein (GFP) from the CDKI-inducible cytomegalovirus (CMV) promoter, followed by subcloning of GFP positive cells and monitoring the induction of GFP expression by IPTG. A cell line showing approximately 10-fold increase in GFP upon the addition of IPTG was used for HTS in a 96-well format. This reporter line was used to screen two diversified small-molecule libraries developed by ChemBridge Corp., Microformat 04 and DiverSet, each comprising 50,000 compounds. These diversified libraries were rationally chosen by ChemBridge by quantifying pharmacophores in a collection of >500,000 drug-like molecules, using a version of Chem-X software to maximize the pharmacophore diversity. The Microformat 04 collection was designed to complement the chemical space covered by the older DiverSet library. The ChemBridge libraries were screened at 20 μM concentration, a conventional concentration for cell-based screening of these libraries. 62 of 100,000 ChemBridge compounds were identified by HTS and verified as inhibiting the induction of CMV-GFP expression in response to p21. This low hit rate (0.06%) indicates a high selectivity of our assay. Structures of 56 of these active compounds are shown in FIG. 1. Active SNX2-class compounds are shown in FIG. 2. Inactive compounds are shown in FIG. 3.

### Example 2

### Effect of Identified Compounds on CDKI-Induced Transcription on Reporter Genes

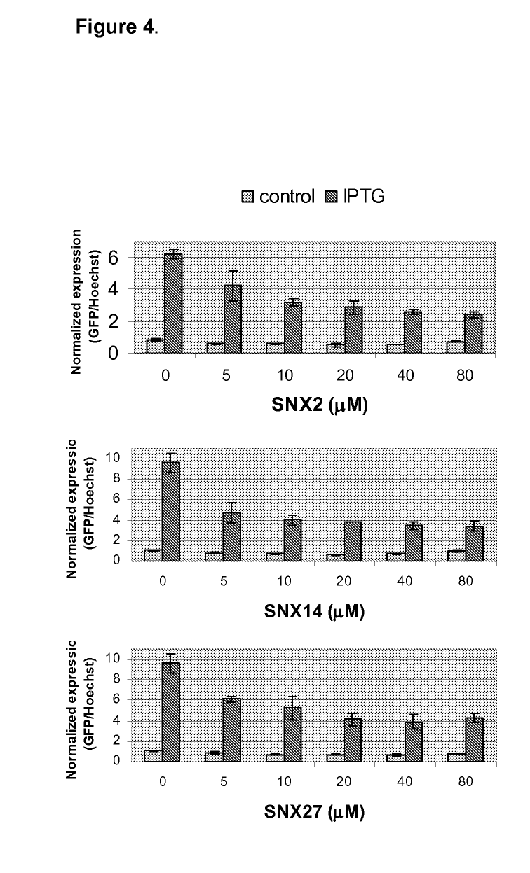

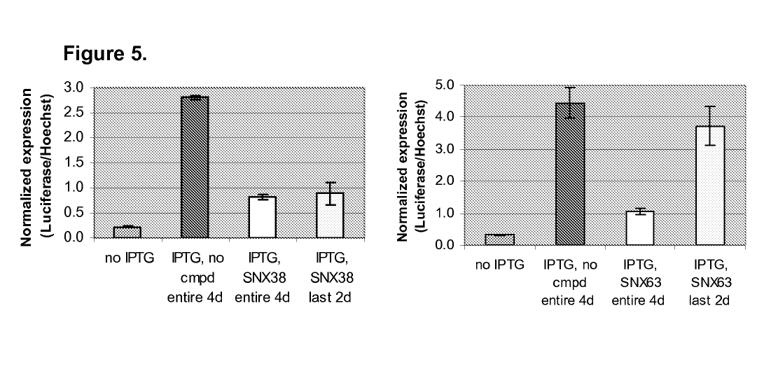

FIG. 4 shows the effects of different doses of some SNX2-class compounds on CMV promoter activity, represented as GFP expression in the reporter cell line from the CMV promoter normalized by cellular DNA content (a measure of cell number) as measured by Hoechst 33342 staining, in the presence or in the absence of IPTG (the p21 inducer). The compounds show pronounced dose-dependent inhibition of transcription by p21, but they have only a marginal effect on the promoter function when p21 is not induced. The experiment in FIG. 5 shows that some SNX2-class compounds not only prevent but also reverse p21-induced transcription. In this experiment, HT1080 p21-9 cells that express firefly luciferase from a CDKI-responsive promoter of cellular NK4 gene were cultured with IPTG for two days, which is sufficient for near-maximal induction of NK4. The addition of SNX2-class compound SNX38 strongly decreased the induction of NK4-luciferase by p21 not only when the compound was added simultaneously with IPTG but also when added after two days of IPTG treatment, indicating that the compound not only prevents but also reverses CDKI-induced transcription. As a negative control, FIG. 5 shows that an unrelated compound SNX63 inhibited transcription only when added simultaneously with IPTG but not two days later. The ability to reverse CDKI-induced transcription suggests that drugs derived from SNX2-class compounds may be useful not only for chemoprevention but also for therapeutic applications.

### Example 3

### Effect of Identified Compounds on CDKI-Induced Transcription on Endogenous Genes

We determined whether SNX2-class compounds inhibit the CDKI effect not only on artificial promoter-reporter constructs but also on CDKI-responsive endogenous genes. For this purpose, we developed real-time reverse-transcription PCR (Q-PCR) assays for measuring RNA levels of eleven CDKI-responsive genes. This assay uses a 96-well TurboCapture RNA extraction kit (Qiagen), in which oligo(dT) is covalently bound to the surface of the wells to allow mRNA isolation from cell lysate and cDNA synthesis in the same wells. 5 units/μl of SuperScript III reverse transcriptase (Invitrogen) was added to the wells for 1 hr for cDNA synthesis at 50° C., and 2 μl of the resulting cDNA was then used for Q-PCR analysis using SYBR Green PCR Master Mix (ABI) with ABI 7900HT Q-PCR machine. Primers used to amplify specific gene products for the corresponding genes and for β-actin (control) are listed in Table 2.

TABLE 2

Sequence of primers used in Q-PCR

| | | | Product |
|---|---|---|---|
| Gene | Sense (5′-3′) | Antisense (5′-3′) | size (bp) |
| Acid β- | CGATCGAGCATATGTTGCTG | AGTTCACACGTCCCATGT | 134 |
| galactosidase | | | |
| CC3 | ATCCGAGCCGTTCTCTACAA | CTGGTGACGCCTCTTGGT | 111 |
| (Complement | | | |
| C3) | | | |
| CTGF | GGAGTGGGTGTGTGACGAG | CCAGGCAGTTGGCTCTAATC | 116 |
| (Connective | | | |
| Tissue Growth | | | |
| Factor) | | | |
| LGALS3 | GGAGCCTACCCTGCCACT | CCGTGCCCAGAATTGTTATC | 118 |
| (Galectin-3, | | | |
| Mac-2) | | | |
| NK4 | CACAGCACCAGGCCATAGA | TCTGCCAGGCTCGACATC | 85 |
| p66shc | TTCGAGTTGCGCTTCAAAC | TCAGGTGGCTCTTCCTCCT | 116 |
| SAA | GTTCCTTGGCGAGGCTTT | CCCCGAGCATGGAAGTATT | 105 |
| SGP | GCTTCCTGCCAGACCCTTAC | CCAATTTTCAAGCACACGAA | 118 |
| (Prosaposin) | | | |
| SOD2 | CCTAACGGTGGTGGAGAACC | CAGCCGTCAGCTTCTCCTTA | 94 |
| βAPP | GGACCAAAACCTGCATTGAT | CTGGATGGTCACTGGTTGG | 113 |
| β-Actin | CTTCCTGGGCATGGAGTC | TGTTGGCGTACAGGTCTTTG | 95 |

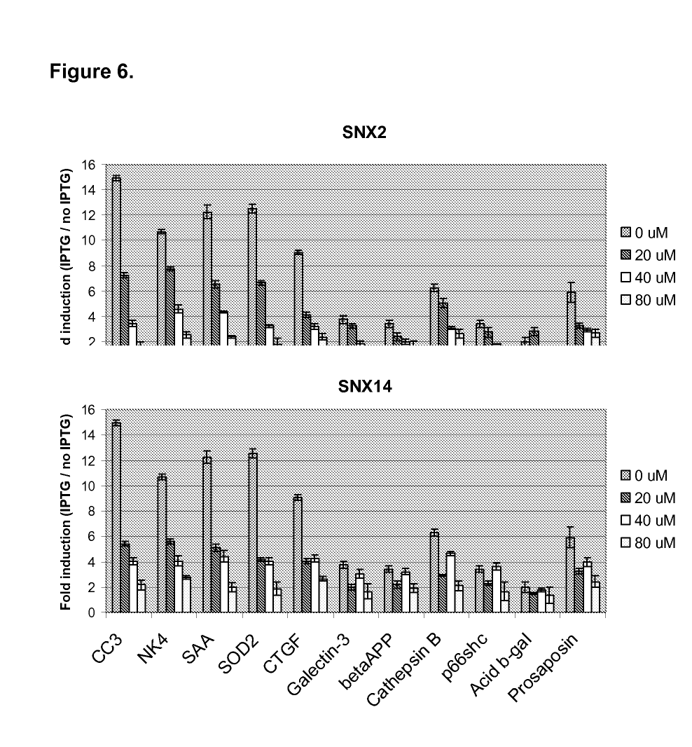

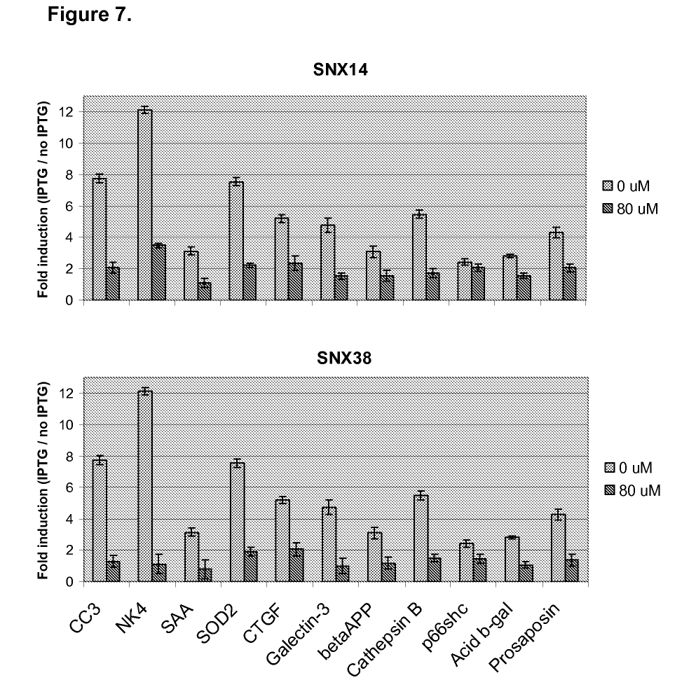

FIGS. 6 and 7 show the data obtained with SNX2 and SNX14, with the results expressed as the ratio of RNA levels for each gene in the presence and in the absence of IPTG (β-actin, expression of which is not affected by CDKI, was used as a normalization standard). This analysis showed that SNX2-class compounds completely or partially inhibit the induction of all the tested genes in cells arrested by CDKI, as shown for p21-arrested cells in FIG. 6 and for p16-arrested cells in FIG. 7. These results argue that the molecular target of SNX2-class compounds is not a specific CDKI but rather a common downstream mediator of the transcription-inducing effects of different CDKI.

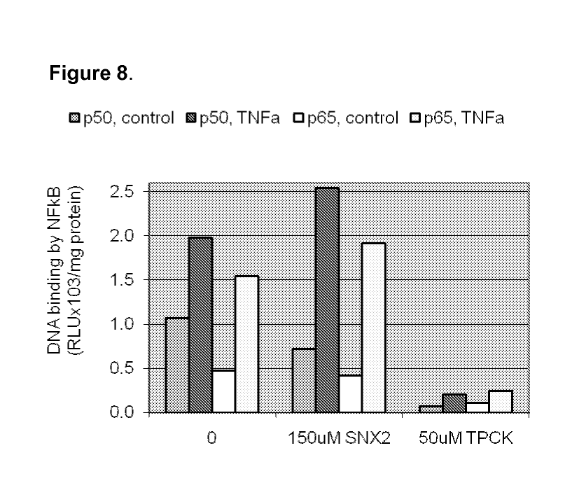

We also tested if these compounds could act as the inhibitors of NFκB, by measuring cellular levels of p50 or p65 subunits binding oligonucleotides containing NFκB consensus binding site, using ACTIVE MOTIF TransAM™ NFκB p65 Chemi and NFκB p50 Chemi Transcription Factor Assay Kits. As shown in FIG. 8, SNX2 has no significant effect on either TNFα-induced or basal NFκB activity, in contrast to NFκB inhibitor TPCK (positive control), which completely blocks NFκB activity in these assays.

### Example 4

### Effects of SNX2-Class Compounds on CDKI-Induced Cell Cycle Arrest

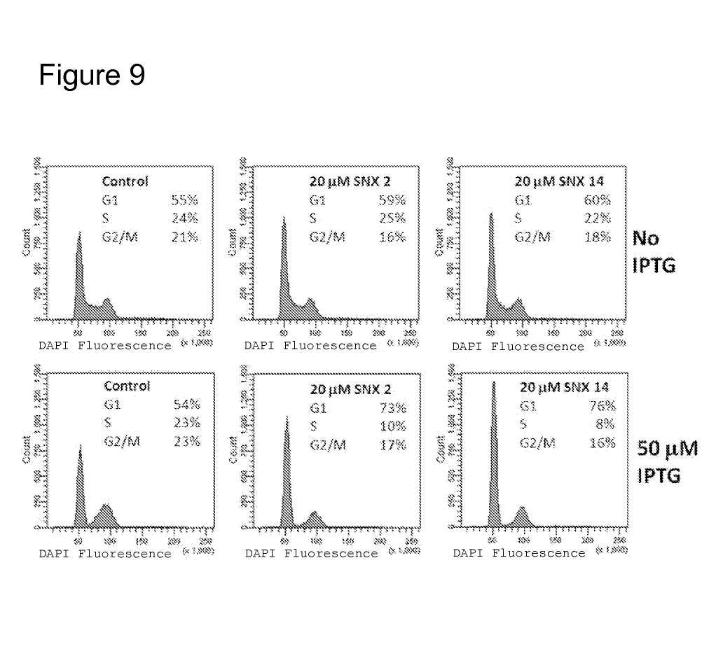

While SNX2-class compounds have a desirable activity of inhibiting the induction of transcription by CDKI proteins, they do not interfere with the tumor-suppressive function of p21 as an inhibitor of cell growth, as indicated by the inability of the compounds to increase cell number upon p21 induction. We have analyzed the effect of SNX2-class compounds on cell cycle distribution of p21-arrested HT1080 p21-9 cells. Upon p21 induction, these cells are known to arrest both in G1 and in G2 (Chang et al., Oncogene 19, 2165-2170), which is illustrated in FIG. 9 by a reduction in the S-phase but not in the G1 or G2 fractions of cells treated with 50 μM IPTG for 18 hrs, relative to IPTG-untreated cells (as determined by FACS analysis of DNA content in DAPI-stained cells). 20 μM concentrations of SNX2 or SNX14 produce a small increase in the G1 fraction in the absence of IPTG (4% increase with SNX2 and 5% increase with SNX14) (FIG. 9). However, when SNX2 and SNX14 were added simultaneously with IPTG, they produced a much greater increase in the G1 fraction relative to cells treated with IPTG alone (19% increase with SNX2 and 22% increase with SNX14) (FIG. 9). While increasing the G1 fraction, SNX2-class compounds concurrently decreased the G2 fraction of IPTG-treated cells (6% decrease with SNX2 and 7% decrease with SNX14) (FIG. 9). Hence, SNX2-class compounds increase p21-induced G1 arrest while decreasing p21-induced G2 arrest.

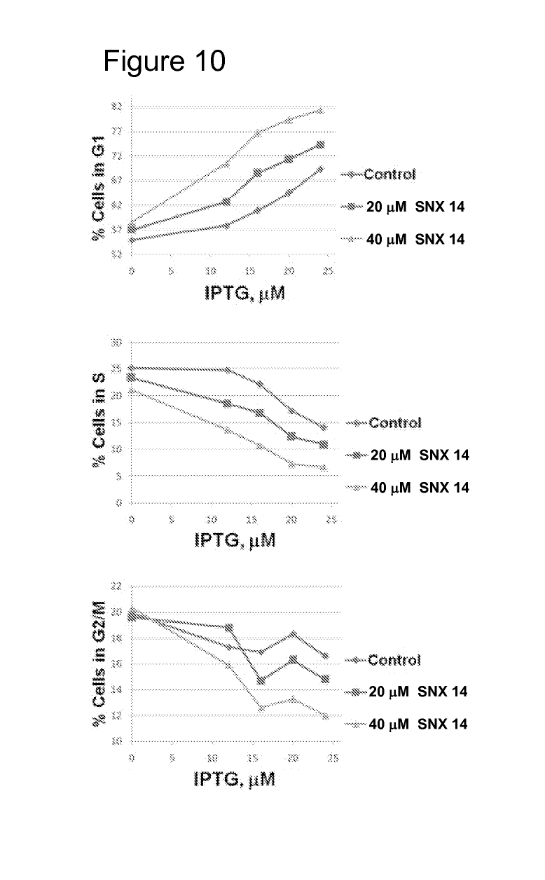

To determine whether the increase in p21-induced G1 arrest represents the primary cell cycle effect of SNX2-class compounds or a secondary consequence of their interference with p21-induced G2 arrest, we have used cell line HT1080 p27-2 with IPTG-inducible expression of the CDKI p27 (CDKN1 B) (Maliyekkel et al, Cell Cycle 5, 2390-2395). p27 is a specific inhibitor of CDK2 (which is also inhibited by p21); unlike p21, p27 induces cell cycle arrest only in G1. FIG. 10 shows the effects of different doses of p27-inducing IPTG on the fraction of cells in G1, S or G2, in the presence of 0, 20 μM or 40 μM SNX14. IPTG induces dose-dependent increase in the G1 fraction with a corresponding decrease in S and G2/M. The doses of IPTG used in this experiment induce G1 arrest at levels that are lower than the maximal levels that are produced by 50-100 μM IPTG, where >80% of cells are in G1. The effect of these lower doses of IPTG that induce detectable but sub-maximal G1 arrest, is strongly augmented by 20 μM and, to an even greater extent, by 40 μM SNX14 (FIG. 10). Hence, SNX2-class compounds increase the G1 arrest activity of CDKI proteins.

These findings offer a mechanism for CDKI pathway inhibition by SNX2-class compounds. CDKI proteins have two distinct activities: (i) they bind to cyclin/CDK complexes, inhibiting their kinase activity and causing cell cycle arrest, and (ii) they activate the CDKI pathway, leading to transcriptional activation of CDKI-responsive genes. SNX2-class CDKI pathway inhibitors diminish CDKI pathway activation by the CDKI proteins by “shifting” the CDKIs towards CDK binding and inhibition. As a result, SNX2-class compounds not only inhibit the CDKI pathway but also enhance the desirable, tumor-suppressive activity of the CDKI proteins as cell cycle inhibitors. The tumor suppression-enhancing activity of SNX2-class CDKI pathway inhibitors indicates their potential utility as cancer chemopreventive agents. The synergistic interaction of these compounds with CDKIs in inducing G1 arrest also indicates their utility as adjuncts to conventional chemotherapeutic drugs or radiation, which arrest tumor cell division by inducing the expression of CDKIs (principally p21).

### Example 5

### Biological Activities of SNX2-Class Compounds

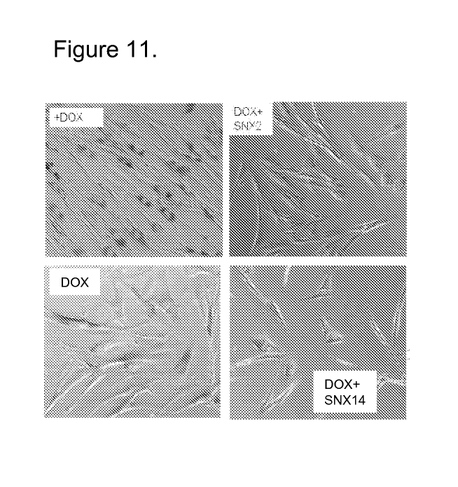

We have correlated the ability of SNX2-class compounds to inhibit the induction of CDKI-responsive genes with their effect on the senescent phenotype, induced in normal human WI-38 fibroblasts by treatment with 200 nM doxorubicin. As shown in FIG. 11, doxorubicin induces expression of the senescence marker SA-β-gal (blue staining), but SNX2 and SNX14 block this phenotype and also diminish morphological changes associated with cell senescence.

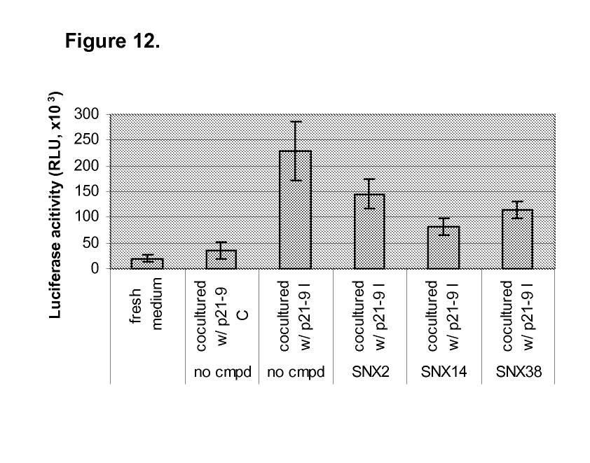

We have also tested if SNX2-class compounds can inhibit paracrine tumor-promoting activities of CDKI-expressing cells. In the assay shown in FIG. 12, HT1080 p21-9 cells were either untreated or treated with p21-inducing IPTG, alone or in the presence of three SNX2-class compounds (SNX2, SNX14 and SNX38). After three days, cells were trypsinized, washed to remove residual compounds, and 3×10³ cell aliquots of each sample were mixed (in 6 replicates) with 10⁴ cell aliquots of C8 mouse fibroblast line, which is highly susceptible to apoptosis in low-serum media. (To detect C8 cells in co-culture, we had transduced them with a vector expressing firefly luciferase.) The next day after plating the mixtures in 96-well plates (in 10% serum and in the absence of IPTG or compounds), cells were exposed to low-serum (0.5%) media, and the relative number of surviving C8 cells was measured after 3 days by the luciferase assay. Cells that underwent p21 induction increased C8 cell survival >5-fold, but this effect was significantly diminished when p21 induction was carried out in the presence of the SNX2-class compounds, with SNX14 showing the strongest effect (FIG. 12).

1. A pharmaceutical formulation comprising the compound SNX-2 or SNX-14 and a pharmaceutically acceptable diluents or carrier.

2. A method for treating a degenerative diseases of the central nervous system in a patient afflicted with a degenerative disease of the central nervous system, comprising administering to the patient a therapeutic amount of a compound having the structure 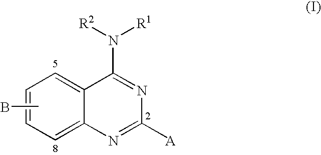
wherein
R¹ is selected from lower alkyl, cycloalkyl, alkenyl, alkynyl, hydroxyalkyl, alkoxyalkyl, hydroxyalkoxyalkyl, dialkylaminoalkyl, aralkyl, aryl, heteroaryl, phenethyl, and alkoxyphenyl;
R² is selected from R¹ and hydrogen;
A is selected from hydrogen or R¹; and
B is halogen.

3. The method according 2 claim 2, wherein, R¹ is selected from C1-C3 alkyl, C2-C3 alkenyl, C2-C3 alkynyl, C7-C8 aralkyl, C2-C3-O-alkyl substituted aryl, and a 3-6 membered heteroalkyl group having 1-2 heteroatoms selected from O and N, wherein R¹ is C2-C3 alkyl when R² is not hydrogen.

4. The method according to claim 3, wherein R² is hydrogen.

5. The method according to claim 3, wherein A is hydrogen.

6. The method according to claim 2, wherein the compound has a structure shown in FIG. 2.

7. The method according to claim 2, wherein the compound is SNX-2 or SNX-14.

8. The method according to claim 2, wherein the degenerative disease is a dementia

9. The method according to claim 8, wherein the dementia is Alzheimer's Disease.


SEQUENCE LISTING (22 sequences)

SEQ ID NO: 1 - DNA, 20 bp, Artificial Sequence
  Description of Artificial Sequence Synthetic primer
cgatcgagcatatgttgctg

SEQ ID NO: 2 - DNA, 20 bp, Artificial Sequence
  Description of Artificial Sequence Synthetic primer
atccgagccgttctctacaa

SEQ ID NO: 3 - DNA, 19 bp, Artificial Sequence
  Description of Artificial Sequence Synthetic primer
ggagtgggtgtgtgacgag

SEQ ID NO: 4 - DNA, 18 bp, Artificial Sequence
  Description of Artificial Sequence Synthetic primer
ggagcctaccctgccact

SEQ ID NO: 5 - DNA, 19 bp, Artificial Sequence
  Description of Artificial Sequence Synthetic primer
cacagcaccaggccataga

SEQ ID NO: 6 - DNA, 19 bp, Artificial Sequence
  Description of Artificial Sequence Synthetic primer
ttcgagttgcgcttcaaac

SEQ ID NO: 7 - DNA, 18 bp, Artificial Sequence
  Description of Artificial Sequence Synthetic primer
gttccttggcgaggcttt

SEQ ID NO: 8 - DNA, 20 bp, Artificial Sequence
  Description of Artificial Sequence Synthetic primer
gcttcctgccagacccttac

SEQ ID NO: 9 - DNA, 20 bp, Artificial Sequence
  Description of Artificial Sequence Synthetic primer
cctaacggtggtggagaacc

SEQ ID NO: 10 - DNA, 20 bp, Artificial Sequence
  Description of Artificial Sequence Synthetic primer
ggaccaaaacctgcattgat

SEQ ID NO: 11 - DNA, 18 bp, Artificial Sequence
  Description of Artificial Sequence Synthetic primer
cttcctgggcatggagtc

SEQ ID NO: 12 - DNA, 18 bp, Artificial Sequence
  Description of Artificial Sequence Synthetic primer
agttcacacgtcccatgt

SEQ ID NO: 13 - DNA, 18 bp, Artificial Sequence
  Description of Artificial Sequence Synthetic primer
ctggtgacgcctcttggt

SEQ ID NO: 14 - DNA, 20 bp, Artificial Sequence
  Description of Artificial Sequence Synthetic primer
ccaggcagttggctctaatc

SEQ ID NO: 15 - DNA, 20 bp, Artificial Sequence
  Description of Artificial Sequence Synthetic primer
ccgtgcccagaattgttatc

SEQ ID NO: 16 - DNA, 18 bp, Artificial Sequence
  Description of Artificial Sequence Synthetic primer
tctgccaggctcgacatc

SEQ ID NO: 17 - DNA, 19 bp, Artificial Sequence
  Description of Artificial Sequence Synthetic primer
tcaggtggctcttcctcct

SEQ ID NO: 18 - DNA, 19 bp, Artificial Sequence
  Description of Artificial Sequence Synthetic primer
ccccgagcatggaagtatt

SEQ ID NO: 19 - DNA, 20 bp, Artificial Sequence
  Description of Artificial Sequence Synthetic primer
ccaattttcaagcacacgaa

SEQ ID NO: 20 - DNA, 20 bp, Artificial Sequence
  Description of Artificial Sequence Synthetic primer
cagccgtcagcttctcctta

SEQ ID NO: 21 - DNA, 19 bp, Artificial Sequence
  Description of Artificial Sequence Synthetic primer
ctggatggtcactggttgg

SEQ ID NO: 22 - DNA, 20 bp, Artificial Sequence
  Description of Artificial Sequence Synthetic primer
tgttggcgtacaggtctttg